(19) United States
(12) Patent Application Publication
(10) Pub. No.: US 2009/0227979 A1
(43) Pub. Date: Sep. 10, 2009

(54) Drug Level Sensor for Injection Device

(76) Inventor: Robert J. Sanchez, JR., Oceanside, CA (US)

Correspondence Address: ALCON, IP LEGAL, TB4-8, 6201 SOUTH FREEWAY, FORT WORTH, TX, 76134, US

(21) Appl. No.: 12/044,405

(22) Filed: Mar. 7, 2008

(51) Int. Cl. A61M 31/00; B67D 5/08

(52) U.S. Cl. 604/506; 222/40; 222/54; 604/113

(57) ABSTRACT

A dispensing chamber assembly has a dispensing chamber housing, a plunger, and a level sensor. The dispensing chamber housing at least partially surrounds a dispensing chamber that holds a quantity of a substance. The plunger is fluidly sealed to the inner surface of the dispensing chamber housing. The level sensor detects when the dispensing chamber is substantially full.

### BACKGROUND OF THE INVENTION

The present invention relates to a single-use medical device and more particularly to a two-piece ophthalmic drug delivery device with a disposable tip end that uses a glass drug chamber.

Several diseases and conditions of the posterior segment of the eye threaten vision. Age related macular degeneration (ARMD), choroidal neovascularization (CNV), retinopathies (e.g., diabetic retinopathy, vitreoretinopathy), retinitis (e.g., cytomegalovirus (CMV) retinitis), uveitis, macular edema, glaucoma, and neuropathies are several examples.

These, and other diseases, can be treated by injecting a drug into the eye. Such injections are typically manually performed using a conventional syringe and needle. FIG. 1 is a perspective view of a prior art syringe used to inject drugs into the eye. In FIG. 1, the syringe includes a needle 105, a luer hub 110, a chamber 115, a plunger 120, a plunger shaft 125, and a thumb rest 130. As is commonly known, the drug to be injected is located in chamber 115. Pushing on the thumb rest 130 causes the plunger 120 to expel the drug through needle 105.

In using such a syringe, the surgeon is required to pierce the eye tissue with the needle, hold the syringe steady, and actuate the syringe plunger (with or without assistance) to inject the fluid into the eye. The volume injected is typically not controlled in an accurate manner because reading the vernier is subject to parallax error. Fluid flow rates are uncontrolled and tissue damage may occur due to an “unsteady” injection. Reflux of the drug may also occur when the needle is removed from the eye.

An effort has been made to control the delivery of small amounts of liquids. A commercially available fluid dispenser is the ULTRA™ positive displacement dispenser available from EFD Inc. of Providence, R.I. The ULTRA dispenser is typically used in the dispensing of small volumes of industrial adhesives. It utilizes a conventional syringe and a custom dispensing tip. The syringe plunger is actuated using an electrical stepper motor and an actuating fluid. With this type of dispenser, the volumes delivered are highly dependent on fluid viscosity, surface tension, and the specific dispensing tip. Parker Hannifin Corporation of Cleveland, Ohio distributes a small volume liquid dispenser for drug discovery applications made by Aurora Instruments LLC of San Diego, Calif. The Parker/Aurora dispenser utilizes a piezo-electric dispensing mechanism. While precise, this dispenser is expensive and requires an electrical signal to be delivered to the dispensing mechanism.

U.S. Pat. No. 6,290,690 discloses an ophthalmic system for injecting a viscous fluid (e.g. silicone oil) into the eye while simultaneously aspirating a second viscous fluid (e.g. perflourocarbon liquid) from the eye in a fluid/fluid exchange during surgery to repair a retinal detachment or tear. The system includes a conventional syringe with a plunger. One end of the syringe is fluidly coupled to a source of pneumatic pressure that provides a constant pneumatic pressure to actuate the plunger. The other end of the syringe is fluidly coupled to an infusion cannula via tubing to deliver the viscous fluid to be injected.

It would be desirable to have a portable hand piece for injecting a drug into the eye. Such a hand piece can include a limited reuse assembly attachable to and removable from a disposable tip segment. Properly dosing and delivering the drug can be challenging. A pre-set dosage of a drug suspended in a phase transition material must be heated before being delivered into the eye. Such heating causes expansion which can result in inaccurate dosing. What is needed is a dosing aid, such as a drug level sensor, that accurately detects the level of the drug in a dispensing chamber.

### SUMMARY OF THE INVENTION

In one embodiment consistent with the principles of the present invention, the present invention is a dispensing chamber assembly comprising a dispensing chamber housing, a plunger, and a level sensor. The dispensing chamber housing at least partially surrounds a dispensing chamber that holds a quantity of a substance. The plunger is fluidly sealed to the inner surface of the dispensing chamber housing. The level sensor detects when the dispensing chamber is substantially full.

In another embodiment consistent with the principles of the present invention, the present invention is a dispensing chamber assembly comprising a dispensing chamber housing, a temperature control device, a plunger, and a pressure or piezoelectric sensor. The dispensing chamber housing at least partially surrounds a dispensing chamber that holds a quantity of a substance and a quantity of air. The temperature control device at least partially surrounds the dispensing chamber housing. The plunger is fluidly sealed to the inner surface of the dispensing chamber housing. The pressure or piezoelectric sensor detects when the substance reaches a specified level within the dispensing chamber and the quantity of air is substantially purged from the dispensing chamber. When the plunger is driven, the quantity of air is substantially purged from the dispensing chamber and the substance contacts the pressure or piezoelectric sensor.

In another embodiment consistent with the principles of the present invention, the present invention is a dispensing chamber assembly comprising a dispensing chamber housing, a temperature control device, a plunger, and a pair of contacts. The dispensing chamber housing at least partially surrounds a dispensing chamber that holds a quantity of a substance and a quantity of air. The temperature control device at least partially surrounds the dispensing chamber housing. The plunger is fluidly sealed to the inner surface of the dispensing chamber housing. The pair of contacts detect when the substance reaches a specified level within the dispensing chamber and the quantity of air is substantially purged from the dispensing chamber. The drug is always in contact with the drug. When the plunger is driven, the quantity of air is substantially purged from the dispensing chamber and the substance touches the second contacts thereby closing an electrical circuit

In another embodiment consistent with the principles of the present invention, the present invention is a method of injecting a drug suspended in a phase transition compound into an eye comprising: altering the temperature of a quantity of a substance and a quantity of air contained in a dispensing chamber; after the temperature of the quantity of the substance and the quantity of the air has been altered, activating an actuator to move a plunger located in the dispensing chamber; expelling a substantial amount of the quantity of air from a dispensing chamber; detecting when the air has been expelled from the dispensing chamber; and activating the actuator a second time to move the plunger to dispense the substance from the dispensing chamber.

It is to be understood that both the foregoing general description and the following detailed description are exemplary and explanatory only and are intended to provide further explanation of the invention as claimed. The following description, as well as the practice of the invention, set forth and suggest additional advantages and purposes of the invention.

### BRIEF DESCRIPTION OF THE DRAWINGS

The accompanying figures, which are incorporated in and constitute a part of this specification, illustrate several embodiments of the invention and together with the description, serve to explain the principles of the invention.

FIG. 1 is a perspective view of a prior art syringe.

FIG. 2 is a view of an ophthalmic medical device including a disposable tip segment and a limited reuse assembly according to the principles of the present invention.

FIG. 3 is an embodiment of a limited reuse assembly according to the principles of the present invention.

FIG. 4 is a cross section view of a disposable tip segment and a limited reuse assembly according to the principles of the present invention.

FIG. 5 is a cross section view of a disposable tip segment according to the principles of the present invention.

FIG. 6 is a cross section view of a dispensing chamber housing assembly according to the principles of the present invention.

FIG. 7 is a cross section view of a dispensing chamber housing assembly according to the principles of the present invention.

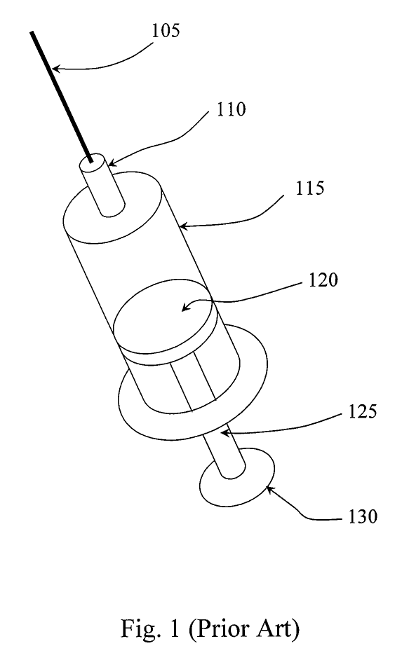

### DETAILED DESCRIPTION OF THE PREFERRED EMBODIMENTS

Reference is now made in detail to the exemplary embodiments of the invention, examples of which are illustrated in the accompanying figures. Wherever possible, the same reference numbers are used throughout the figures to refer to the same or like parts.

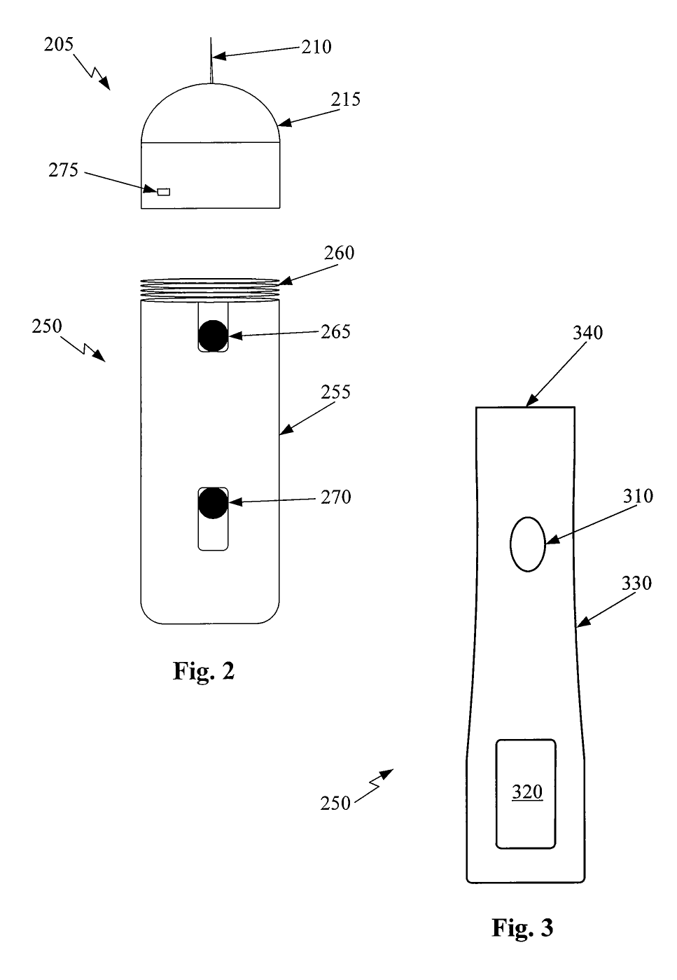

FIG. 2 depicts one view of an ophthalmic medical device including a disposable tip segment and a limited reuse assembly according to an embodiment of the present invention. In FIG. 2, the medical device includes a tip segment 205 and a limited reuse assembly 250. The tip segment 205 includes a needle 210, a housing 215, and an optional light 275. The limited reuse assembly 250 includes a housing 255, a switch 270, a lock mechanism 265, and a threaded portion 260.

Tip segment 205 is capable of being connected to and removed from limited reuse assembly 250. In this embodiment, tip segment 205 has a threaded portion on an interior surface of housing 215 that screws onto the threaded portion 260 of limited reuse assembly 250. In addition, lock mechanism 265 secures tip segment 215 to limited reuse assembly 250. Lock mechanism 265 may be in the form of a button, a sliding switch, or a cantilevered mechanism. Other mechanisms for connecting tip segment 205 to limited reuse assembly 250, such as those involving structural features that mate with each other, are commonly known in the art and are within the scope of the present invention.

Needle 210 is adapted to deliver a substance, such as a drug, into an eye. Needle 210 may be of any commonly known configuration. Preferably, needle 210 is designed such that its thermal characteristics are conducive to the particular drug delivery application. For example, when a heated drug is to be delivered, needle 210 may be relatively short (several millimeters) in length to facilitate proper delivery of the drug based on thermal characteristics.

Switch 270 is adapted to provide an input to the system. For example, switch 270 may be used to activate the system or to turn on a heater. Other switches, buttons, or user-directed control inputs are commonly known and may be employed with limited reuse assembly 250 and/or tip segment 205.

Optional light 275 is illuminated when tip segment 205 is ready to be used. Optional light 275 may protrude from housing 215, or it may be contained within housing 215, in which case, optional light 275 may be seen through a clear portion of housing 215. In other embodiments, optional light 275 may be replaced by an indicator, such as a liquid crystal display, segmented display, or other device that indicates a status or condition of disposable tip segment 205. For example, optional light 275 may also pulse on and off to indicate other states, such as, but not limited to a system error, fully charged battery, insufficiently charged battery or faulty connection between the tip segment 205 and limited use assembly 250. While shown on tip segment 205, optional light 275 or other indicator may be located on limited reuse assembly 250.

FIG. 3 is another embodiment of a limited reuse assembly according to the principles of the present invention. Limited reuse assembly 250 includes a button 310, a display 320, and a housing 330. Disposable tip segment 205 attaches to end 340 of limited reuse assembly 250. Button 310 is actuated to provide an input to the system. As with switch 270, button 310 may activate a heater or other temperature control device or initiate actuation of a plunger. Display 320 is a liquid crystal display, segmented display, or other device that indicates a status or condition of disposable tip segment 205 or limited reuse assembly 250.

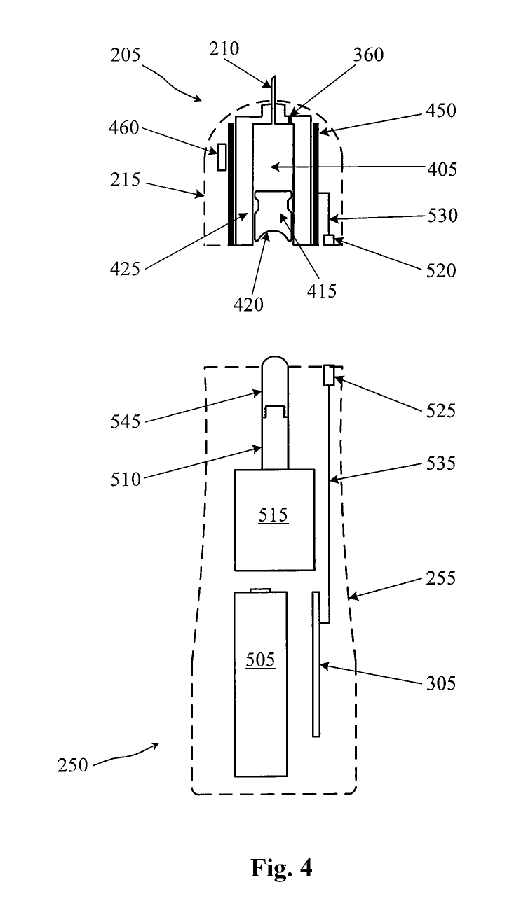

FIG. 4 is a cross section view of a disposable tip segment and a limited reuse assembly according to an embodiment of the present invention. FIG. 4 shows how tip segment 205 interfaces with limited reuse assembly 250. In the embodiment of FIG. 4, tip segment 205 includes plunger interface 420, plunger 415, level sensor 360, dispensing chamber housing 425, tip segment housing 215, temperature control device 450, thermal sensor 460, needle 210, dispensing chamber 405, interface 530, and tip interface connector 520. Limited reuse assembly 250 includes mechanical linkage 545, actuator shaft 510, actuator 515, power source 505, controller 305, limited reuse assembly housing 255, interface 535, and limited reuse assembly interface connector 525.

Needle 210 is fluidly coupled to dispensing chamber 405. In such a case, a substance contained in dispensing chamber 405 can pass through needle 210 and into an eye. Temperature control device 450 at least partially surrounds dispensing chamber housing 425. In this case, temperature control device 450 is adapted to heat and/or cool dispensing chamber housing 425 and any substance contained in dispensing chamber 405. Interface 530 connects temperature control device 450 with tip interface connector 520.

Level sensor 360 is embedded in or located on dispensing chamber housing 425. Level sensor 360 functions to detect the presence of a substance in dispensing chamber 405. This detection generally occurs when the substance contacts level sensor 360. For example, level sensor 360 may be a piezoelectric sensor. As is known, a piezoelectric sensor is a device that uses the piezoelectric effect to measure pressure by convereting pressure into an electrical signal. In this case, a quantity of a substance can be placed in dispensing chamber 405. The dispensing chamber 405 can also contain air in addition to the substance. Plunger 415 can be moved, expelling air from needle 210, until the substance conatcts level sensor 360. When the substance applies pressure to level sensor 360, an electrical signal is produced that indicates that dispensing chamber 405 is full (and that the substance has filled dispensing chamber 415 thereby making the device ready for an injection). While described as a piezoelectric sensor, any number of pressure or force sensors may be employed as level sensor 360.

For ophthalmic injections, a phase transition compound/drug mixture may be injected into the eye. Such a mixture expands when heated to a less viscous state. When it is heated (and in a less viscous state) it can be injected into the eye. After heating, plunger 415 is advanced until the mixture contacts level sensor 360. When level sensor 360 detects the mixture, the plunger is stopped, and the mixture is ready for injection. In such a case, dispensing chamber 405 is substantially full of the mixture. It should be noted that such an approach is particularly desirable because not all of the air needs to be expelled from dispensing chamber 405 before an injection occurs. In other words, injecting a small quantity of air into the eye produces no harm and can better facilitate an injection of a phase transition compound/drug mixture. In such a case, keeping air in the needle prevents it from clogging before an injection.

In limited reuse assembly 250, power source 505 provides power to actuator 515. An interface (not shown) between power source 505 and actuator 515 serves as a conduit for providing power to actuator 515. Actuator 515 is connected to actuator shaft 510. When actuator 515 is a stepper motor, actuator shaft 510 is integral with actuator 515. Mechanical linkage interface 545 is connected to actuator shaft 510. In this configuration, as actuator 515 moves actuator shaft 510 upward toward needle 210 mechanical linkage interface 545 also moves upward toward needle 210.

Controller 305 is connected via interface 535 to limited reuse assembly interface connecter 525. Limited reuse assembly interface connecter 525 is located on a top surface of limited reuse assembly housing 255 adjacent to mechanical linkage interface 545. In this manner, both limited reuse assembly interface connector 525 and mechanical linkage interface 545 are adapted to be connected with tip interface connector 520 and plunger interface 420 respectively.

Controller 305 and actuator 515 are connected by an interface (not shown). This interface (not shown) allows controller 305 to control the operation of actuator 515. In addition, an interface (not shown) between power source 505 and controller 305 allows controller 305 to control operation of power source of 310. In such a case, controller 305 may control the charging and the discharging of power source 505 when power source 505 is a rechargeable battery.

Controller 305 is typically an integrated circuit with power, input, and output pins capable of performing logic functions. In various embodiments, controller 305 is a targeted device controller. In such a case, controller 305 performs specific control functions targeted to a specific device or component, such as a temperature control device or a power supply. For example, a temperature control device controller has the basic functionality to control a temperature control device. In other embodiments, controller 305 is a microprocessor. In such a case, controller 305 is programmable so that it can function to control more than one component of the device. In other cases, controller 305 is not a programmable microprocessor, but instead is a special purpose controller configured to control different components that perform different functions. While depicted as one component, controller 305 may be made of many different components or integrated circuits.

Tip segment 205 is adapted to mate with or attach to limited reuse assembly 250 as previously described. In the embodiment of FIG. 4, plunger interface 420 located on a bottom surface of plunger 415 is adapted to mate with mechanical linkage interface 545 located near a top surface of limited reuse assembly housing 255. In addition, tip interface connector 520 is adapted to connect with limited reuse assembly interface connector 525. When tip segment 205 is connected to limited reuse assembly 250 in this manner, actuator 515 and actuator shaft 510 are adapted to drive plunger 415 upward toward needle 210. In addition, an interface is formed between controller 305 and temperature control device 450. A signal can pass from controller 305 to temperature control device 450 through interface 535, limited reuse assembly interface connector 525, tip interface connector 520, and interface 530.

In operation, when tip segment 205 is connected to limited reuse assembly 250, controller 305 controls the operation of actuator 515. Actuator 515 is actuated and actuator shaft 510 is moved upward toward needle 210. In turn, mechanical linkage interface 545, which is mated with plunger interface 420, moves plunger 415 upward toward needle 210. A substance located in dispensing chamber 405 is then expelled through needle 210.

In addition, controller 305 controls the operation of temperature control device 450. Temperature control device 450 is adapted to heat and/or cool dispensing chamber housing 425. Since dispensing chamber housing 425 is at least partially thermally conductive, heating or cooling dispensing chamber housing 425 heats or cools a substance located in dispensing chamber 405. Temperature information can be transferred from thermal sensor 460 to controller 305 via any of a number of different interface configurations. This temperature information can be used to control the operation of temperature control device 450. When temperature control device 450 is a heater, controller 305 controls the amount of current that is sent to temperature control device 450. The more current sent to temperature control device 450, the hotter it gets. In such a manner, controller 305 can use a feed back loop utilizing information from thermal sensor 460 to control the operation of temperature control device 450. Any suitable type of control algorithm, such as a proportional integral derivative (PID) algorithm, can be used to control the operation of temperature control device 450.

Typically, for an ophthalmic injection, tip segment 205 is connected to limited reuse assembly 250. Temperature control device 450 is activated to bring the substance to within a proper temperature range or to a proper temperature. Actuator 515 drives plunger 415 to expel air from dispensing chamber 405. Level sensor 360 detects when the substance in dispensing chamber 405 reaches a known position (and the air in dispensing chamber 405 is substantially purged from it). Actuator 515 stops, and plunger 415 remains in place. The device is now ready for an injection. A doctor inserts needle 210 into the eye and depresses a button to start actuator 515. Actuator 515 then moves plunger 415 a precise distance to deliver a precise dosage into the eye.

In one embodiment of the present invention, the substance located in dispensing chamber 405 is a drug that is preloaded into dispensing chamber 405. When a drug is preloaded into dispensing chamber 405, a set quantity of the drug can be preloaded. For example, 100 microliters of a drug can be loaded into dispensing chamber 405, and any quantity up to 100 microliters can be dispensed. In such a case, the plunger 415 can be moved a precise distance to deliver a precise dosage of drug from the dispensing chamber 405, through the needle 210, and into an eye. This provides for flexibility of dosing and for ease of assembly.

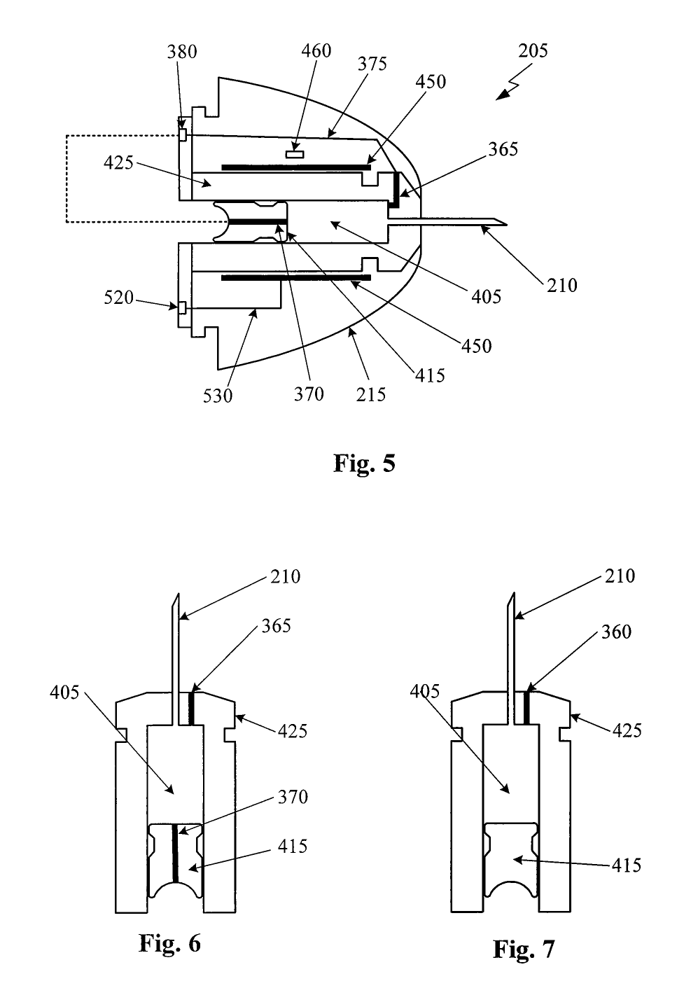

FIG. 5 is a cross section view of a disposable tip segment for an ophthalmic medical device according to an embodiment of the present invention. In FIG. 5, disposable tip segment 205 includes housing 215, needle 210, dispensing chamber 405, dispensing chamber housing 425, plunger 415, first contact 370, second contact 365, temperature control device 450, thermal sensor 460, interface 375, level sensor interface 380, interface 530, and tip interface connector 520. Disposable tip segment 205 operates as a disposable injection device.

In the embodiment of FIG. 5, needle 210 is fluidly coupled to dispensing chamber 405. Temperature control device 450 at least partially surrounds dispensing chamber housing 425 and dispensing chamber 405. Housing 215 forms an outer skin on disposable tip segment 205.

In various embodiments of the present invention, temperature control device 450 is a heating and/or a cooling device. Temperature control device 450 is in thermal contact with dispensing chamber housing 425. As such, temperature control device 450 is capable of changing the temperature of the substance in dispensing chamber 405. Interface 530 and tip interface connector 520 couple temperature control device 450 to a limited reuse assembly. In such a case, temperature control device 450 can be powered and controlled by the limited reuse assembly.

A substance to be delivered into an eye, typically a drug, is located in dispensing chamber 405. Typically, dispensing chamber 405 is cylindrical in shape. Temperature control device 450 is in thermal contact with dispensing chamber housing 425. In this manner, temperature control device 450 is adapted to control the temperature of the contents of dispensing chamber 405. Thermal sensor 460 provides temperature information to assist in controlling the operation of temperature control device 450.

In this embodiment of the present invention, the level sensor comprises two contacts, 365 and 370, interface 375, and level sensor interface 380. When the substance in dispensing chamber 405 is at least partially electrically conductive, a simple level sensor design includes two contacts—365 and 370. When the substance in dispensing chamber 405 touches both contacts 365 and 370, an electrical current can pass through the substance (in effect closing a switch—the electrical path is shown by the dotted line). This electrical current is easily detected (just as the closing of a switch is easily detected). Again, it should be noted that such an approach is particularly desirable because not all of the air needs to be expelled from dispensing chamber 405 before an injection occurs. In other words, injecting a small quantity of air into the eye produces no harm and can better facilitate an injection of a phase transition compound/drug mixture. In such a case, keeping air in the needle prevents it from clogging before an injection.

In this embodiment, second contact 365 is embedded in or located on dispensing chamber housing 425. First contact 370 is located in or on plunger 415. Typically, the first and second contacts, 365 and 370, are simple electrical contacts that can be made of any of a number of different materials that are electrically conductive. The first and second contacts, 365 and 370, are located such that a substance located in dispensing chamber 405 can touch both contacts when plunger 415 is moved pushing the substance within the chamber to a specified level.

FIG. 6 is a cross section view of a dispensing chamber housing assembly according to the principles of the present invention. In FIG. 6, plunger 415 is located in dispensing chamber housing 425. Dispensing chamber 405 is bounded by the interior surface of dispensing chamber housing 425 and a face of plunger 415. Needle 210 is fluidly coupled to dispensing chamber 405. Second contact 365 is embedded in or located on dispensing chamber housing 425. First contact 370 is embedded in or located on plunger 415. First and second contacts, 365 and 370, form a level sensor as described in FIG. 5.

FIG. 7 is a cross section view of dispensing chamber housing assembly according to the principles of the present invention. In FIG. 6, plunger 415 is located in dispensing chamber housing 425. Dispensing chamber 405 is bounded by the interior surface of dispensing chamber housing 425 and a face of plunger 415. Needle 210 is fluidly coupled to dispensing chamber 405. Level sensor 360 is embedded in or located on dispensing chamber housing 425 as described in FIG. 4.

From the above, it may be appreciated that the present invention provides an improved system for delivering precise volumes of a substance into an eye. The present invention provides a single use, disposable delivery device tip segment that is capable of delivering a precise dosage. The tip segment interfaces with a limited reuse assembly. The disposable tip segment has a level sensor that detects the level of a substance to be dispensed. The present invention is illustrated herein by example, and various modifications may be made by a person of ordinary skill in the art.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1-11. (canceled)

12. A dispensing chamber assembly comprising:
a dispensing chamber housing at least partially surrounding a dispensing chamber, the dispensing chamber for holding a quantity of a substance and a quantity of air;
a plunger fluidly sealed to the inner surface of the dispensing chamber housing; and
first and second electrical contacts for determining when the dispensing chamber is substantially full and the quantity of air is substantially, but not completely, purged from the dispensing chamber;
wherein the substance touches the first and second contacts thereby closing an electrical circuit to indicate that the quantity of air is substantially, but not completely, purged from the dispensing chamber.

13. The assembly of claim 19 wherein the temperature control device is a heater for heating a drug suspended in a phase transition compound.

14. The assembly of claim 12 further comprising:
a thermal sensor in thermal contact with the dispensing chamber housing

15. A method of injecting a drug suspended in a phase transition compound into an eye, the method comprising:
altering the temperature of a quantity of a substance and a quantity of air contained in a dispensing chamber;
after the temperature of the quantity of the substance and the quantity of the air has been altered, activating an actuator to move a plunger located in the dispensing chamber; expelling a substantial amount of the quantity of air from a dispensing chamber;
detecting when the substance has reached a specific location; and
activating the actuator a second time to move the plunger to dispense the substance from the dispensing chamber.

16. The method of claim 15 wherein altering the temperature of a quantity of a substance and a quantity of air contained in a dispensing chamber further comprises:
heating the quantity of the substance and the quantity of the air so that the quantity of the substance and the quantity of the air both expand.

17. The assembly of claim 12 wherein the first contact is located on the plunger and the second contact is located on the dispensing chamber housing.

18. The assembly of claim 12 wherein the first and second electrical contacts are located such that a quantity of air remains in the needle prior to an injection.

19. The assembly of claim 12 further comprising:
a temperature control device at least partially surrounding the dispensing chamber housing.
